(19) United States
(12) Patent Application Publication
(10) Pub. No.: US 2010/0191274 A1
(43) Pub. Date: Jul. 29, 2010

(54) FILTER DEPLOYMENT DEVICE

(75) Inventors: Ari Ryan, Mountain View, CA (US); Ali Salahieh, Saratoga, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/360,563

(22) Filed: Jan. 27, 2009

(51) Int. Cl. A61B 17/22

(52) U.S. Cl. 606/200

(57) ABSTRACT

This invention provides a filter device attached to a guidewire, tube, or catheter and a device for deploying the filter. The deployment device employs heating to activate a shape memory material moving a free end of a latch with respect to the guide wire, tube, or catheter thereby releasing the filter from a containment element. The actuation mechanism of the deployment device is well suited to the deployment of certain filter elements as well as other medical devices.

### TECHNICAL FIELD

This disclosure relates generally to a filter device attached to a guide wire, tube, or catheter and a device for deploying the filter. The actuation mechanism of the deployment device is well suited to the deployment of certain filter elements as well as other medical devices.

### Background

Human blood vessels often become occluded or blocked by plaque, thrombi, other deposits, or material that reduce the blood carrying capacity of the vessel. Should the blockage occur at a critical place in the circulatory system, serious and permanent injury, and even death, can occur. To prevent this, some form of medical intervention is usually performed when significant occlusion is detected.

Several procedures are now used to open these stenosed or occluded blood vessels in a patient caused by the deposit of plaque or other material on the walls of the blood vessels. Angioplasty, for example, is a widely known procedure wherein an inflatable balloon is introduced into the occluded region. The balloon is inflated, dilating the occlusion, and thereby increasing the intraluminal diameter.

Another procedure is atherectomy. During atherectomy, a catheter is inserted into a narrowed artery to remove the matter occluding or narrowing the artery, i.e., fatty material. The catheter includes a rotating blade or cutter disposed in the tip thereof. Also located at the tip are an aperture and a balloon disposed on the opposite side of the catheter tip from the aperture. As the tip is placed in close proximity to the fatty material, the balloon is inflated to force the aperture into contact with the fatty material. When the blade is rotated, portions of the fatty material are shaved off and retained within the interior lumen of the catheter. This process is repeated until a sufficient amount of fatty material is removed and substantially normal blood flow is resumed.

In another procedure, stenosis within arteries and other blood vessels is treated by permanently or temporarily introducing a stent into the stenosed region to open the lumen of the vessel. The stent typically includes a substantially cylindrical tube or mesh sleeve made from such materials as stainless steel or nitinol. The design of the material permits the diameter of the stent to be radially expanded, while still providing sufficient rigidity such that the stent maintains its shape once it has been enlarged to a desired size.

Unfortunately, such percutaneous interventional procedures, i.e., angioplasty, atherectomy, and stenting, often dislodge material from the vessel walls. Some existing devices and technology use a filter for capturing the dislodged material from the bloodstream.

### SUMMARY

There still is a need for filter devices having a low profile delivery mechanism that is easily activated remotely and which is readily adaptable to a variety of activation environments. Accordingly, the present disclosure provides a filter device and associated deployment apparatus configured to be used in connection with an intravascular device such as a catheter or guidewire. The filter deployment apparatus includes a filter element, a support structure for the filter element, a containment element, the containment element having one or more apertures, and a heat-activatable latch, wherein the heat-activatable latch is operable between a first position and a second position by heating a portion of the latch. In the first position, the latch is engaged with one or more apertures of the containment element and in the second position it is disengaged with at least a majority of the one or more apertures of the containment element.

The heat-activatable latch of the disclosure is operable between a first position and a second position by the application of heat to at least a portion of the heat-activatable latch. The latch includes a means to heat at least a portion of the latch, preferably electrically or through thermal contact with a heated fluid. Generally, the heat-activated latch includes a shape memory material which reverts to a former shape when heated, moving from a first position to a second position thereby removing a portion of the latch from one or more apertures, and either directly deploying a medical device, such as a filter, or allowing a medical device such as a filter to deploy.

The disclosure may include a method of deploying a filter disposed on a guide wire, tube, or catheter by providing a containment element, the containment element having one or more apertures therein and a heat-activatable latch. Heating at least a portion of the heat-activatable latch initially engaged with one or more of the apertures in the containment element causes the heat-activatable latch to disengage from apertures in the containment element releasing one or both of the filter element and the support structure for the filter element. The heat-activatable latch may be used in a similar manner to release a stent from a containment element or to activate other medical devices.

### BRIEF DESCRIPTION OF DRAWINGS

FIGS. 1A and 1B are a simplified guide tube bearing a heat-activatable retractable latch in extended and retracted positions, respectively.

FIGS. 2A and 2B are an alternate version of a distal portion of a guide tube bearing a heat-activatable retractable latch in extended and retracted positions, respectively.

FIGS. 3A and 3B are an alternate version of a distal portion of a guide tube bearing a heat-activatable retractable latch in extended and retracted positions, respectively.

FIGS. 4A and 4B are an alternate version of a distal portion of a guide tube bearing a retractable latch and a filter within a containment device in engaged and disengaged positions, respectively.

FIGS. 5A and 5B are an alternate version of a guide tube bearing a heat-activatable retractable latch and a filter wherein the latch is in extended and retracted positions, respectively.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

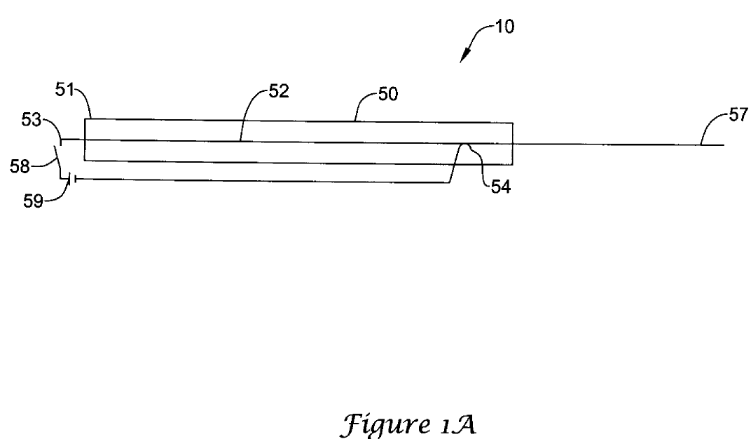

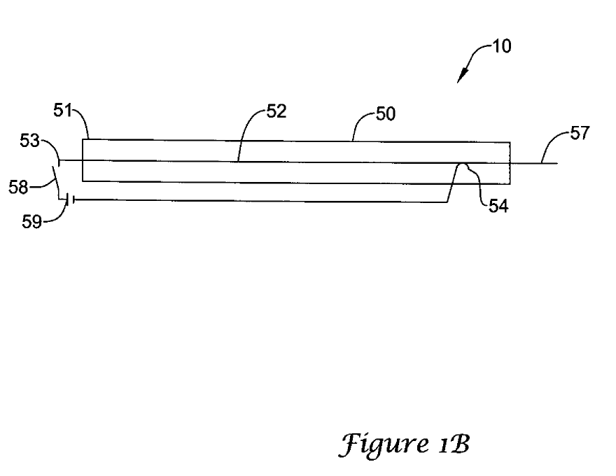

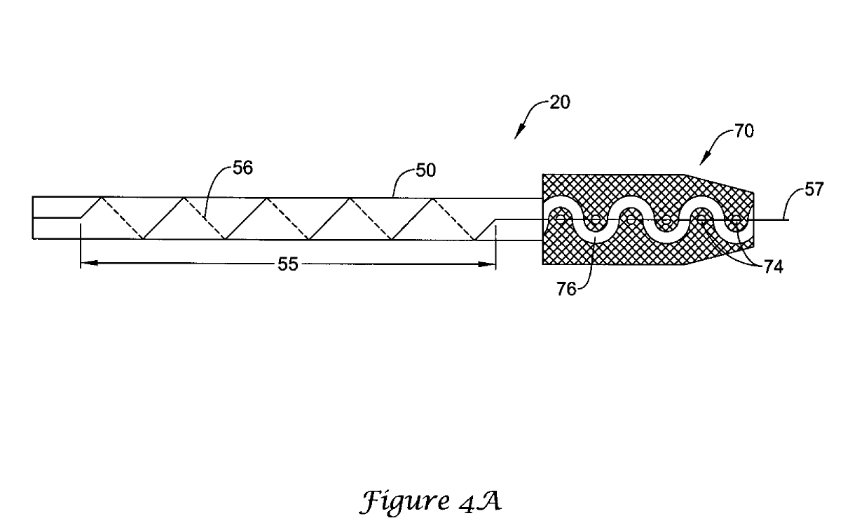

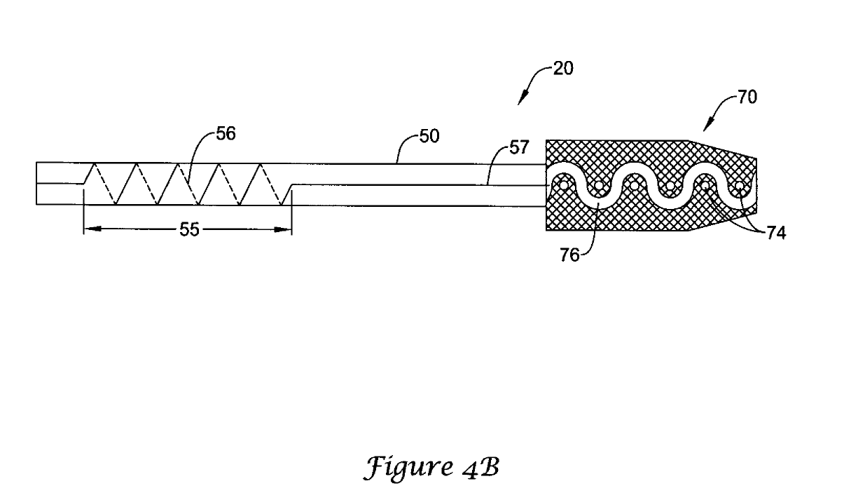

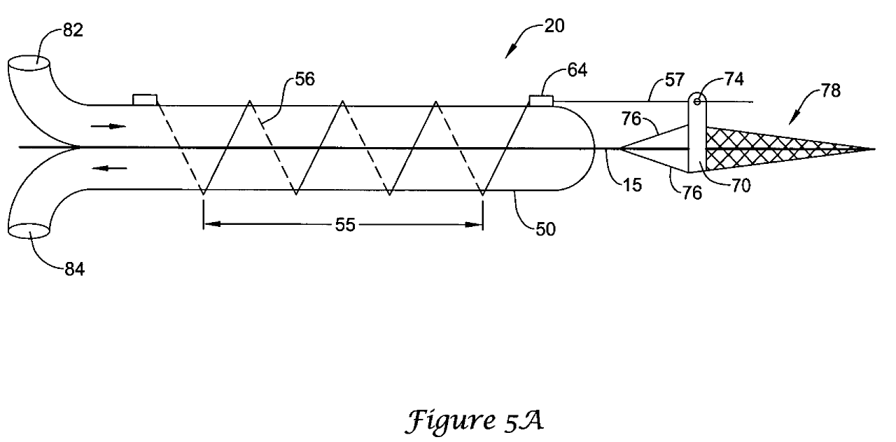

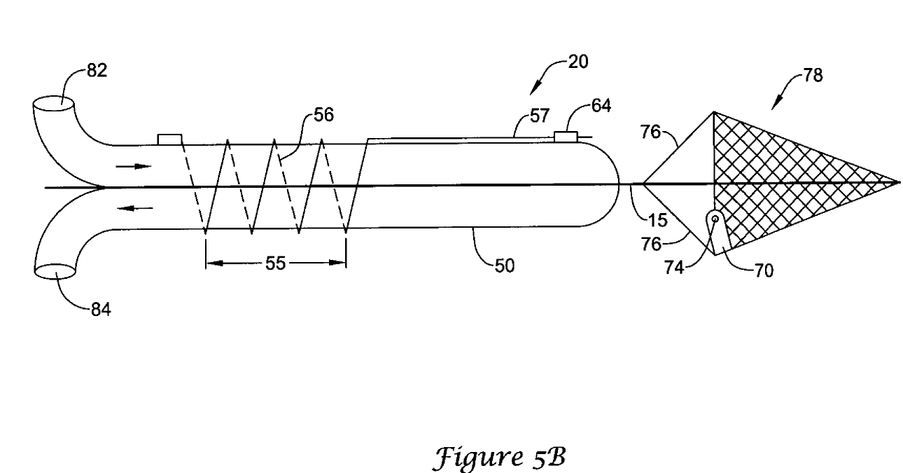

FIGS. 1-3 illustrate several example configurations of the latch mechanism of the disclosure. The filter, containment device, or other medical device which would be associated with the distal end of the guide tube, catheter, or second guide wire has been omitted for clarity although it will be understood that they will be configured to engage with the latch in a first position and to disengage from the latch in a second position as illustrated in greater detail in the embodiments of FIGS. 4 and 5. Although nitinol will be used as the shape-memory material throughout the description below, it will be appreciated that other shape memory materials may be used instead of, or in conjunction with, nitinol.

In the schematic FIGS. 1A and 1B, latch mechanism 10 comprising a stretched linear nitinol wire 52 is deployed alongside or within support member 50. The distal end 57 of nitinol wire latch element 52 initially extends beyond the support member 50 prior to heating as seen in FIG. 1A. Heating nitinol wire 52, for example by supplying an electrical current through the wire by means of a contact 53, switch 58, and current source 59 near the proximal end 51 of the support member and a return path through contact 54 situated near the distal end of the nitinol wire, causes the wire to return to its previously unstretched length thereby retracting the wire relative to the distal end of the support member 50 in FIG. 1B. It will be appreciated that the distal end of the nitinol wire or latch element 57 need not extend beyond the distal end of the support member 50, but has been presented in that configuration for illustrative purposes. In some embodiments, it may be desirable to coat all or a portion of the nitinol wire to provide electrical isolation and/or to control heating or cooling of the nitinol wire 52.

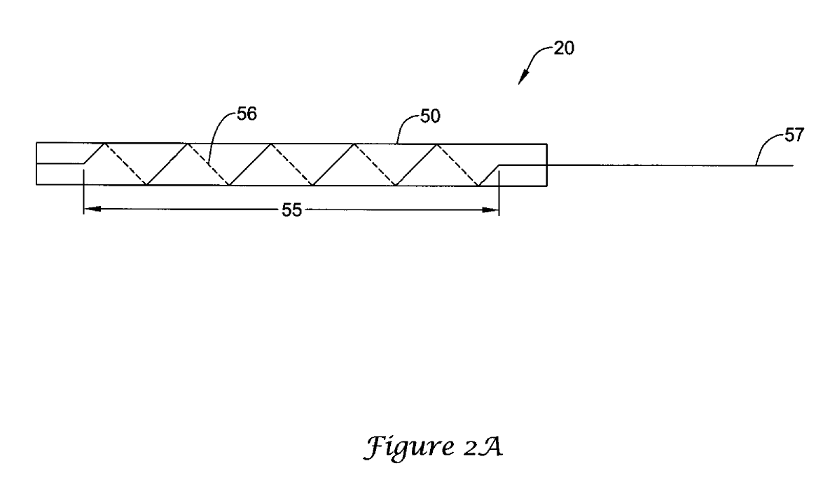

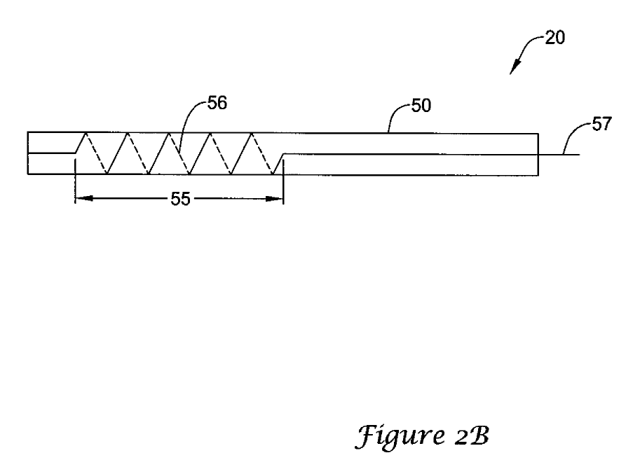

FIGS. 2A and 2B present an alternate configuration of the latch mechanism 20 in which a portion of the nitinol latch element 56 is wound around support member 50. Although latch element 56 is depicted as a generally circular cross-section wire wound as a helix, it should be apparent that other forms such as a flat ribbon may be used with similar results and the choice of shape may be subject to other design considerations such as relative lateral flexibility or stiffness. The latch element 56 comprises a helical region 55. Depending on the design of the remainder of the latch mechanism and the associated medical device, either of the states of FIG. 2A or 2B may represent the initial cooled state when either of FIG. 2B or 2A represents the corresponding final state attained after heating. In the first case, the latch mechanism 20 retracts the distal end 57 of the nitinol latch element 56 upon heating (not shown), while in the second case, the latch mechanism extends the distal end 57 of the nitinol latch element 56 upon heating.

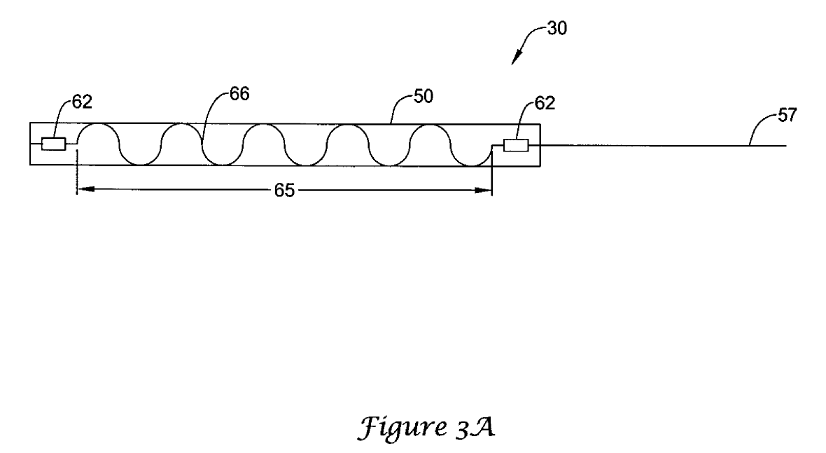

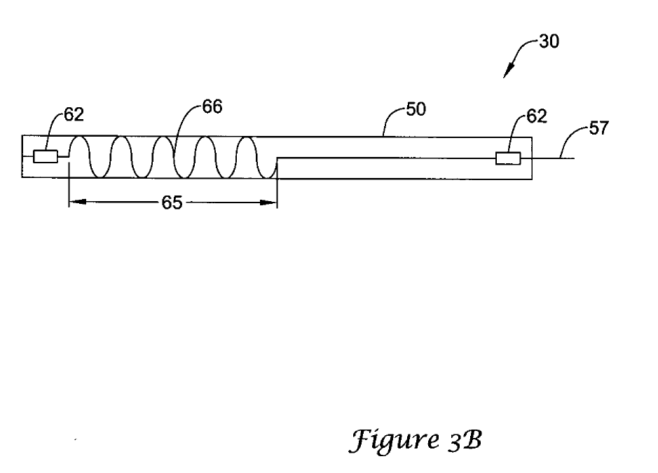

In FIGS. 3A and 3B, the nitinol latch element 66 of latch mechanism 30 is formed in a multiply-bent configuration. As illustrated, the multiply-bent configuration is generally sinusoidal although other periodic or aperiodic configurations are contemplated. In addition, the configuration may be substantially two-dimensional, may include a twist, or may conform to an interior or exterior surface of support member 50.

In the configurations of FIGS. 3A and 3B, the latch mechanisms 30 are shown with optional guides 62,64 associated with a support member 50. The optional guides typically will direct the motion of the latch element 66 in a desired direction, for example, parallel to the axis of the support member such that distal tip 67 moves generally along support member 50. In some configurations, guides 62,64 may be mounted on the surface of support member 50 while in other configurations the guides 62,64 as well as the latch element 66 may reside within a lumen of support member 50. In some embodiments, the guides may be apertures in the wall of a support member 50 and portions of the latch element may reside within a lumen of support member 50 while other portions lie outside of support member 50. In yet other embodiments, the guides 62,64 may occupy positions staggered around the circumference of the support element to provide a circumferential rotation or spiral component to the motion of the distal tip 67. In electrically heated embodiments, one or both of the guides 62,64 may provide an electrical contact with latch element 66. As in FIGS. 2A and 2B, either of FIGS. 3A and 3B may represent the low temperature state of region 65 leading to contracted or extended heated states respectively.

Although not illustrated, other configurations are possible. It will be appreciated that the latch element may be wound in a spiral about a support member and include a locked-in torque such that upon heating, the nitinol latch member tip describes a spiral path and may engage or disengage with a threaded or thread-like portion of the medical device. Alternatively, the spiral arrangement may provide a torque to engage or disengage the medical device.

FIGS. 4A and 4B are illustrative of the use of a heat-activated latch mechanism 20 which may be used to release a filter and filter support structure (not shown) from a containment element 70 having a plurality of apertures 74 on either side of a parting line 76. In FIG. 4A, distal end 57 of nitinol latch element 56 has been threaded through apertures 74 on alternating sides of the parting line to fix the containment element 70 in a closed position until the latch element tip 57 is withdrawn. Heating at least region 55 of latch element 56 electrically, by thermal contact with a heated fluid or body, or the like causes the region 55 to return to an unstretched shape as shown in FIG. 4B. This withdraws distal tip 57 from the apertures 74 allowing containment element 70 to open, thereby releasing and deploying the filter and its associated support structure.

In FIGS. 5A and 5B, heat-activated latch mechanism 20 includes nitinol latch element 56 wrapped around support member 50 which includes a fluid circuit between inlet 82 and outlet 84 which typically are located near the proximal end of the support member. Passing a heated liquid or gas through the fluid circuit warms support member 50 and thus region 55 of latch element 56 causing the stretched region 55 of FIG. 5A to return to the unstretched state of FIG. 5B thereby withdrawing a portion of the distal region of the latch element 56 through guide 64 as distal tip 57 retracts from apertures 74 of containment element 70. In the illustrated embodiment, release of containment element 70 allows outwardly biased struts 76 to deploy filter 78 about support wire 15.

Details of the heating arrangement can include, for example, a second, conductive wire may be attached to the nitinol latch element near its distal end or the contact may be provided by a wiper. In such embodiments both the nitinol wire and the conductive electrical return path can be insulated. A power source attached to the proximal ends of the nitinol wire and an adjacent conductive wire may supply current through the conductive wire causing the nitinol to heat and return to its unstretched state. In some embodiments, an insulated nitinol wire exposed at its distal end may suffice to provide a complete electrical circuit if the body and bodily fluids are capable of providing a return path carrying the current necessary to heat the nitinol wire. In some embodiments, a portion of the nitinol element may be locally thinned to confine heating primarily to a region of interest. In yet other embodiments, the nitinol element may comprise only a relatively short segment of the latch element, the remainder being formed of more conductive materials such as copper, to generally confine the electrical heating to the nitinol segment. In further embodiments, the nitinol latch element may be attached, near its distal or proximal end, to a guide tube, catheter, or second guide wire which provides at least a portion of a complete electrical circuit through the nitinol latch element.

As indicated earlier, heat may also be supplied to the nitinol latch element, or a portion thereof, by heating an adjacent portion of the medical device delivery apparatus. For example, heated fluid may be circulated through the lumen of a catheter adjacent to the nitinol latch element. Alternatively, the fluid may be heated locally by an immersed heating coil within the catheter.

In some embodiments, the filter apparatus includes elements that at least partially envelope a filter element and/or the support structure for the filter element in a collapsed state when the heat-activatable latch is in the first, engaged position and least partially release the filter element and/or the support structure for the filter element when the heat-activatable latch is in the second, disengaged position. Generally the heat-activatable latch is in a first position prior to application of heat to at least a portion of the heat-activatable latch and in a second position following application of heat to at least a portion of the heat-activatable latch.

In some embodiments, the heat-activatable latch may move from a first position to a second position following application of heat to at least a portion of the heat-activatable latch. Heat may be applied to the heat-activatable latch electrically, by direct or indirect conductive contact with a heated fluid, and the like. In some embodiments, the heat-activatable latch is self-heating when an electric current is passed through at least a portion of the latch or an associated structure. In certain embodiments, at least a portion of the heat-activatable latch comprises a shape memory material, typically nitinol.

In some embodiments more than one heat-activatable latch may be associated with a support member and a medical device or devices. For example, one heat-activatable-latch may deploy a filter and a second heat-activatable latch may deploy or assist in the deployment of a stent from the same catheter. Two or more latches may cooperate to release or otherwise activate a medical device. For example, one latch may release a containment element associated with a filter element and a second latch may release a second containment element associated with a support structure for a filter element.

Various modifications and alterations of this invention will become apparent to those skilled in the art without departing from the scope and principles of the claimed invention, and it should be understood that the claimed invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. A filter deployment apparatus configured to be used in connection with an intravascular device, the filter deployment apparatus comprising:
a supporting element selected from one or more of a catheter, tube, or guide wire;
a filter element associated with the supporting element;
a support structure for the filter element;
a containment element, the containment element having one or more apertures therein; and
a heat-activatable latch having a first position and a second position, wherein the heat-activatable latch is operable between a first position and a second position by heating at least a portion of the heat-activatable latch, further wherein the latch is engaged with the one or more apertures of the containment element in the first position and disengaged with at least a majority of the one or more apertures of the containment element in the second position.

2. The filter deployment apparatus of claim 1, wherein the containment element at least partially envelopes at least one of the filter element and the support structure for the filter element in a collapsed state when the heat-activatable latch is in the first, engaged position.

3. The filter deployment apparatus of claim 1, wherein the containment element at least partially releases the filter element or the support structure for the filter element when the heat-activatable latch is in the second, disengaged position.

4. The filter deployment apparatus of claim 1, wherein the heat-activatable latch is in the first position prior to application of heat to at least a portion of the heat-activatable latch.

5. The filter deployment apparatus of claim 1, wherein the heat-activatable latch is in the second position following application of heat to at least a portion of the heat-activatable latch.

6. The filter deployment apparatus of claim 5, wherein the application of heat is accomplished electrically.

7. The filter deployment apparatus of claim 6, wherein the heat-activatable latch is self-heating when an electric current is passed through at least a portion of the heat-activatable latch.

8. The filter deployment apparatus of claim 5, wherein heat is applied to the heat-activatable latch by conductive contact with a heated fluid.

9. The filter deployment apparatus of claim 1, wherein at least a portion of the heat-activatable latch comprises a shape memory material.

10. The filter deployment apparatus of claim 9, wherein at least a portion of the shape memory material is in a substantially linear configuration.

11. The filter deployment apparatus of claim 9, wherein at least a portion of the shape memory material is in a coiled or multiply-bent configuration.

12. The filter deployment apparatus of claim 1, wherein the first and second positions of the heat-activatable latch lie substantially along a line parallel to the axis of the support element.

13. A heat-activatable latch associated with a support member comprising:
at least one engagement aperture;
a heat-activatable latch, wherein the heat-activatable latch is operable between a first position and a second position, further wherein the latch is engaged with the at least one engagement aperture in the first position and disengaged with at least one engagement aperture in the second position; and
a means to apply heat to the heat-activatable latch.

14. The heat-activatable latch of claim 13 comprises a shape memory material.

15. The heat-activatable latch of claim 13 wherein the motion of the latch is substantially linear.

16. The heat-activatable latch of claim 13, wherein the latch engages a plurality of engagement apertures in the first position.

17. The heat-activatable latch of claim 13, wherein the latch is disengaged from a plurality of engagement apertures in the second position.

18. The heat-activatable latch of claim 13, wherein the heat-activatable latch is in the first position prior to application of heat to at least a portion of the heat-activatable latch.

19. The heat-activatable latch of claim 13, wherein the heat-activatable latch is in the second position following application of heat to at least a portion of the heat-activatable latch.

20. The heat-activatable latch of claim 13, wherein the application of heat is accomplished electrically.

21. The heat-activatable latch of claim 20, wherein the heat-activatable latch is self-heating when an electric current is passed through at least a portion of the heat-activatable latch.

22. The heat-activatable latch of claim 19, wherein the application of heat is accomplished by conductive contact with a heated fluid.

23. The heat-activatable latch of claim 13, wherein the first and second positions of the heat-activatable latch lie substantially along a line parallel to the axis of the catheter or guidewire.

24. The heat-activatable latch of claim 13, wherein at least a portion of the shape memory material is in a substantially linear configuration.

25. The heat-activatable latch of claim 13, wherein at least a portion of the shape memory material is in a coiled or multiply-bent configuration.

26. The heat-activatable latch of claim 13, wherein the engagement aperture is associated with a medical device.

27. The heat-activatable latch of claim 26, wherein the engagement aperture is associated with a filter.

28. The heat-activatable latch of claim 27, wherein operating the heat-activatable latch between the first and second positions deploys the filter.

29. The heat-activatable latch of claim 26, wherein the engagement aperture is associated with a stent.

30. The heat-activatable latch of claim 27, wherein operating the heat-activatable latch between the first and second positions deploys the stent.

31. A method of deploying a filter disposed on a guide wire, tube, or catheter comprising:
providing a catheter, tube, or guidewire;
providing a filter element associated with the catheter, tube, or guidewire;
providing a support structure for the filter element;
providing a containment element, the containment element having one or more apertures therein;
providing a heat-activatable latch and heating at least a portion of the heat-activatable latch engaged with one or more of the apertures in the containment element thereby causing the heat-activatable latch to disengage from the apertures in the containment element releasing at least one of the filter element and the support structure for the filter element.

32. The method of claim 31 wherein heating at least a portion of the heat-activated latch is accomplished electrically.

33. The method of claim 31 wherein heating at least a portion of the heat-activated latch is accomplished by thermal contact with a fluid heated element adjacent the heat-activated latch.

34. The method of claim 31 wherein the filter is replaced by a stent or other medical device.
